(19) United States
(12) Patent Application Publication
(10) Pub. No.: US 2011/0305699 A1
(43) Pub. Date: Dec. 15, 2011

(54) Treating Oral Cancer with Anti-IL-20 Antibody

(75) Inventor: Ming-Shi Chang, Tainan (TW)

(73) Assignee: National Cheng Kung University, Tainan (TW)

(21) Appl. No.: 12/815,927

(22) Filed: Jun. 15, 2010

(51) Int. Cl. A61K 39/395; A61P 35/00

(52) U.S. Cl. 424/135.1; 424/158.1; 424/133.1; 424/145.1

(57) ABSTRACT

Treating oral cancer with an anti-IL-20 antibody.

### BACKGROUND OF THE INVENTION

Oral cancer refers to cancerous tissue growth at a part of the mouth or throat (e.g., lip, tongue, cheek, floor of the mouth, hard and soft palate, sinuses, and pharynx). It can be life-threatening if not diagnosed and treated at an early stage.

### SUMMARY OF THE INVENTION

In one aspect, the present invention features a method for treating oral cancer by administering to a subject in need thereof an effective amount of an anti-IL-20 antibody. In one example, the subject is an oral cancer patient suffering from or at risk for cancer metastasis.

The anti-IL-20 antibody to be used in the method of this invention can be a naturally-occurring antibody, an antigen-binding fragment thereof, or a genetically engineered antibody (e.g., a humanized antibody, a chimeric antibody, or a single-chain antibody). It can contain a heavy chain variable region (V_{H}) including all of the complementarity-determining regions (CDRs) in the V_{H} of monoclonal antibody mAb7E (SEQ ID NO:4) and a light chain variable region (V_{L}) including all of the CDRs in the V_{L} of mAb7E (SEQ ID NO:8). In one example, it is an antibody containing SEQ ID NO:4 and SEQ ID NO:8 (e.g., mAb7E or an antigen-binding fragment thereof).

As used herein, the term “treating” refers to the application or administration of a composition including an anti-IL-20 antibody to a subject, who has oral cancer, a symptom of the cancer, or a predisposition toward the cancer, with the purpose to cure, heal, alleviate, relieve, alter, remedy, ameliorate, improve, or affect the disease, the symptoms of the disease, or the predisposition toward the disease. “An effective amount” as used herein refers to the amount of each active agent required to confer therapeutic effect on the subject, either alone or in combination with one or more other active agents. Effective amounts vary, as recognized by those skilled in the art, depending on route of administration, excipient choice, and co-usage with other active agents.

Also within the scope of this invention is a pharmaceutical composition containing an anti-IL-20 antibody for use in treating oral cancer and use of the pharmaceutical composition for the manufacture of a medicament for oral cancer treatment.

The details of one or more embodiments of the invention are set forth in the description below. Other features or advantages of the present invention will be apparent from the following drawings and detailed description of several examples, and also from the appended claims.

### BRIEF DESCRIPTION OF THE DRAWINGS

The drawings are first described.

FIG. 1 is a diagram showing the effect of anti-IL-20 antibody mAb7E in inhibiting OC-3 oral cancer cell proliferation and migration. A: inhibition of oral cancer cells by mAb7E. B: inhibition of oral cancer cell migration by mAb7E. Values shown in this figure refer to mean±SD. *: P<0.05 (compared to IL-20 treatment).

FIG. 2 is a diagram showing the effect of mAb7E in reducing tumor sizes in mice carrying oral cancer xenogragts. *: P<0.05 (treated mice versus healthy controls).

### DETAILED DESCRIPTION OF THE INVENTION

Disclosed herein is a method for treating oral cancer with an anti-IL-20 antibody. An anti-IL-20 antibody is a naturally-occurring antibody, an antigen-binding fragment thereof, or a genetically engineered antibody that neutralizes IL-20, i.e., binding to IL-20 and blocking the IL-20 mediated signaling pathway.

Naturally-occurring anti-IL-20 antibodies, either polyclonal or monoclonal, can be prepared by conventional methods, using an IL-20 protein or a fragment thereof. See, e.g., Harlow and Lane, (1988) Antibodies: A Laboratory Manual, Cold Spring Harbor Laboratory, New York. A “monoclonal antibody” refers to a homogenous antibody population and a “polyclonal antibody” refers to a heterogenous antibody population. These two terms do not limit the source of an antibody or the manner in which it is made.

IL-20 is a member of the IL-10 cytokine family. Human IL-20 is described under GenBank Accession Number NP_{—}061194 (protein) and NM_{—}018724 (gene).

To produce antibodies against IL-20, the protein or a fragment thereof can be coupled to a carrier protein, such as KLH, mixed with an adjuvant, and injected into a host animal. Antibodies produced in the animal can then be purified by IL-20/IL-20 peptide affinity chromatography. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, CpG, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

Polyclonal antibodies are present in the sera of the immunized subjects. Monoclonal antibodies can be prepared using standard hybridoma technology (see, for example, Kohler et al. (1975) Nature 256, 495; Kohler et al. (1976) Eur. J. Immunol. 6, 511; Kohler et al. (1976) Eur J Immunol 6, 292; and Hammerling et al. (1981) Monoclonal Antibodies and T Cell Hybridomas, Elsevier, N.Y.). In particular, monoclonal antibodies can be obtained by any technique that provides for the production of antibody molecules by continuous cell lines in culture such as described in Kohler et al. (1975) Nature 256, 495 and U.S. Pat. No. 4,376,110; the human B-cell hybridoma technique (Kosbor et al. (1983) Immunol Today 4, 72; Cole et al. (1983) Proc. Natl. Acad. Sci. USA 80, 2026, and the EBV-hybridoma technique (Cole et al. (1983) Monoclonal Antibodies and Cancer Therapy, Alan R. Liss, Inc., pp. 77-96). Such antibodies can be of any immunoglobulin class including IgG, IgM, IgE, IgA, IgD, and any subclass thereof. The hybridoma producing the monoclonal antibodies of the invention may be cultivated in vitro or in vivo. The ability to produce high titers of monoclonal antibodies in vivo makes it a particularly useful method of production. After obtaining antibodies specific to IL-20, their ability to neutralize IL-20 can be determined by a routine procedure.

Fully human anti-IL-20 antibodies, such as those expressed in transgenic animals are also features of the invention. See, e.g., Green et al., Nature Genetics 7:13 (1994), and U.S. Pat. Nos. 5,545,806 and 5,569,825.

Antigen-binding fragments (e.g., F(ab′)₂, Fab, or Fv) of naturally-occurring anti-IL-20 antibodies can be generated by known techniques. For example, F(ab′)₂ fragments can be produced by pepsin digestion of an antibody molecule and Fab fragments can be generated by reducing the disulfide bridges of F(ab′)₂ fragments.

The anti-IL-20 antibody to be used in this invention can also be a genetically engineered antibody, e.g., a humanized antibody, a chimeric antibody, a single chain antibody (scFv), or a domain antibody (dAb; see Ward, et. Al., 1989, Nature, 341:544-546).

A humanized antibody contains a human immunoglobulin (i.e., recipient antibody) in which regions/residues responsible for antigen binding (i.e., the CDRs, particularly the specific-determining residues therein) are replaced with those from a non-human immunoglobulin (i.e., donor antibody). In some instances, one or more residues inside a frame region of the recipient antibody are also replaced with those from the donor antibody. A humanized antibody may also contain residues from neither the recipient antibody nor the donor antibody. These residues are included to further refine and optimize antibody performance. Antibodies can also be humanized by methods known in the art, e.g., recombinant technology.

A chimeric antibody is a molecule in which different portions are derived from different animal species, such as those having a variable region derived from a murine monoclonal antibody and a human immunoglobulin constant region. Such an antibody can be prepared via routine techniques described in, e.g., Morrison et al. (1984) Proc. Natl. Acad. Sci. USA 81, 6851; Neuberger et al. (1984) Nature 312, 604; and Takeda et al. (1984) Nature 314:452.

A single-chain antibody can be prepared via recombinant technology by linking a nucleotide sequence coding for a V_{H} chain and a nucleotide sequence coding for a V_{L} chain. Preferably, a flexible linker is incorporated between the two variable regions. Alternatively, techniques described for the production of single chain antibodies (U.S. Pat. Nos. 4,946,778 and 4,704,692) can be adapted to produce a phage scFv library and scFv clones specific to IL-20 can be identified from the library following routine procedures. Positive clones can be subjected to further screening to identify those that suppress IL-20 activity.

In one example, the anti-IL-20 antibody is monoclonal antibody mAb7E or a functional variant thereof mAb7E is produced by the hybridoma cell line deposited at the American Type Culture Collection, 10801 University Boulevard, Manassas, Va. 20110-2209, U.S.A. and assigned a deposit number PTA-8687. See U.S. Pat. No. 7,435,800 and US 20090048432. This hybridoma cell line will be released to the public irrevocably and without restriction/condition upon granting a US Patent on this application, and will be maintained in the ATCC for a period of at least 30 years from the date of the deposit for the enforceable life of the patent or for a period of 5 years after the date of the most recent. The amino acid sequences/cDNA sequences of the heavy and light chains of mAb7E are shown below.

### Nucleotide Sequence (SEQ ID NO:1) and Amino Acid Sequence (SEQ ID NO:2) of mAb 7E Heavy Chain

| atg tac ttg gga ctg aac tat gta ttc ata gtt ttt ctc tta aat | |
|---|---|
| M Y L G L N Y V F I V F L L N | 15 |
| ggt gtc cag agt gaa ttg aag ctt gag gag tct gga gga ggc ttg | |
| G V Q S E L K L E E S G G G L | 30 |
| gtg cag cct gga gga tcc atg aaa ctc tct tgt gct gcc tct gga | |
| V Q P G G S M K L S C A A S G | 45 |
| ttc act ttt agt gac gcc tgg atg gac tgg gtc cgc cag tct cca | |
| F T F S D A W M D W V R Q S P | 60 |
| gag aag ggg ctt gag tgg att gct gaa att aga agc aaa gct aat | |
| E K G L E W I A E I R S K A N | 75 |
| aat tat gca aca tac ttt gct gag tct gtg aaa ggg agg ttc acc | |
| N Y A T Y F A E S V K G R F T | 90 |
| atc tca aga gat gat tcc aaa agt ggt gtc tac ctg caa atg aac | |
| I S R D D S K S G V Y L Q M N | 105 |
| aac tta aga gct gag gac act ggc att tat ttc tgt acc aag tta | |
| N L R A E D T G I Y F C T K L | 120 |
| tca cta cgt tac tgg ttc ttc gat gtc tgg ggc gca ggg acc acg | |
| S L R Y W F F D V W G A G T T | 135 |
| gtc acc gtc tcc tca gcc aaa acg aca ccc cca tct gtc tat cca | |
| V T V S S A K T T P P S V Y P | 150 |
| ctg gcc cct gga tct gct gcc caa act aac tcc atg gtg acc ctg | |
| L A P G S A A Q T N S M V T L | 165 |
| gga tgc ctg gtc aag ggc tat ttc cct gag cca gtg aca gtg acc | |
| G C L V K G Y F P E P V T V T | 180 |
| tgg aac tct gga tcc ctg tcc agc ggt gtg cac acc ttc cca gct | |
| W N S G S L S S G V H T F P A | 195 |
| gtc ctg cag tct gac ctc tac act ctg agc agc tca gtg act gtc | |
| V L Q S D L Y T L S S S V T V | 210 |
| ccc tcc agc acc tgg ccc agc gag acc gtc acc tgc aac gtt gcc | |
| P S S T W P S E T V T C N V A | 225 |
| cac ccg gcc agc agc acc aag gtg gac aag aaa att gtg ccc agg | |
| H P A S S T K V D K K I V P R | 240 |
| gat tgt ggt tgt aag cct tgc ata tgt aca gtc cca gaa gta tca | |
| D C G C K P C I C T V P E V S | 255 |
| tct gtc ttc atc ttc ccc cca aag ccc aag gat gtg ctc acc att | |
| S V F I F P P K P K D V L T I | 270 |
| act ctg act cct aag gtc acg tgt gtt gtg gta gac atc agc aag | |
| T L T P K V T C V V V D I S K | 285 |
| gat gat ccc gag gtc cag ttc agc tgg ttt gta gat gat gtg gag | |
| D D P E V Q F S W F V D D V E | 300 |
| gtg cac aca gct cag acg caa ccc cgg gag gag cag ttc aac agc | |
| V H T A Q T Q P R E E Q F N S | 315 |
| act ttc cgc tca gtc agt gaa ctt ccc atc atg cac cag gac tgg | |
| T F R S V S E L P I M H Q D W | 330 |
| ctc aat ggc aag gag ttc aaa tgc agg gtc aac agt gca gct ttc | |
| L N G K E F K C R V N S A A F | 345 |
| cct gcc ccc atc gag aaa acc atc tcc aaa acc aaa ggc aga ccg | |
| P A P I E K T I S K T K G R P | 360 |
| aag gct cca cag gtg tac acc att cca cct ccc aag gag cag atg | |
| K A P Q V Y T I P P P K E Q M | 375 |
| gcc aag gat aaa gtc agt ctg acc tgc atg ata aca gac ttc ttc | |
| A K D K V S L T C M I T D F F | 390 |
| cct gaa gac att act gtg gag tgg cag tgg aat ggg cag cca gcg | |
| P E D I T V E W Q W N G Q P A | 405 |
| gag aac tac aag aac act cag ccc atc atg gac aca gat ggc tct | |
| E N Y K N T Q P I M D T D G S | 420 |
| tac ttc gtc tac agc aag ctc aat gtg cag aag agc aac tgg gag | |
| Y F V Y S K L N V Q K S N W E | 435 |
| gca gga aat act ttc acc tgc tct gtg tta cat gag ggc ctg cac | |
| A G N T F T C S V L H E G L H | 450 |
| aac cac cat act gag aag agc ctc tcc cac tct cct ggt aaa TGA | |
| N H H T E K S L S H S P G K — | 464 |

The bold-faced region refers to the V_{H} of mAb 7E heavy chain (DNA sequence SEQ ID NO:3; protein sequence SEQ ID NO:4)

### Nucleotide Sequence (SEQ ID NO:5) and Amino Acid Sequence (SEQ ID NO:6) of mAb 7E Light Chain

| atg atg agt cct gcc cag ttc ctg ttt ctg tta gtg ctc tgg att | |
|---|---|
| M M S P A Q F L F L L V L W I | 15 |
| cgg gaa acc aac ggt gat ttt gtg atg acc cag act cca ctc act | |
| R E T N G D F V M T Q T P L T | 30 |
| ttg tcg gtt acc att gga caa cca gcc tcc atc tct tgc aag tca | |
| L S V T I G Q P A S I S C K S | 45 |
| agt cag agc ctc ttg gat agt gat gga aag aca tat ttg aat tgg | |
| S Q S L L D S D G K T Y L N W | 60 |
| ttg tta cag agg cca ggc cag tct cca aag cac ctc atc tat ctg | |
| L L Q R P G Q S P K H L I Y L | 75 |
| gtg tct aaa ctg gac tct gga gtc cct gac agg ttc act ggc agt | |
| V S K L D S G V P D R F T G S | 90 |
| gga tca ggg acc gat ttc aca ctg aga atc agc aga gtg gag gct | |
| G S G T D F T L R I S R V E A | 105 |
| gag gat ttg gga gtt tat tat tgc tgg caa agt aca cat ttt ccg | |
| E D L G V Y Y C W Q S T H F P | 120 |
| tgg acg ttc ggt gga ggc acc aag ctg gaa atc aaa cgg gct gat | |
| W T F G G G T K L E I K R A D | 135 |
| gct gca cca act gta tcc atc ttc cca cca tcc agt gag cag tta | |
| A A P T V S I F P P S S E Q L | 150 |
| aca tct gga ggt gcc tca gtc gtg tgc ttc ttg aac aac ttc tac | |
| T S G G A S V V C F L N N F Y | 175 |
| aag tgg aag att gat ggc agt gaa cga caa aat ggc gtc ctg aac | |
| P K D I N V K W K I D G S E R | 180 |
| agt tgg act gat cag ccc aaa gac atc aat gtc gac agc aaa gac | |
| Q N G V L N S W T D Q D S K D | 195 |
| agc acc tac agc atg agc agc acc ctc acg ttg acc aag gac gag | |
| S T Y S M S S T L T L T K D E | 210 |
| tat gaa cga cat aac agc tat acc tgt gag gcc act cac aag aca | |
| Y E R H N S Y T C E A T H K T | 225 |
| tca act tca ccc att gtc aag agc ttc aac agg aat gag tgt tag | |
| S T S P I V K S F N R N E C — | 239 |

The bold-faced region refers to the V_{L} of mAb 7E light chain (DNA sequence SEQ ID NO:7; protein sequence SEQ ID NO:8).

A functional variant of mAb7E contains a V_{H} at least 75% (80%, 85%, 90%, or 95%) identical to that of mAb7E (SEQ ID NO:4) and a V_{L} at least 75% (80%, 85%, 90%, or 95%) identical to that of mAb7E (SEQ ID NO:8). As used herein, “percent homology” of two amino acid sequences is determined using the algorism described in Karlin and Altschul, Proc, Natl. Acad. Sci. USA 87:2264-2268, 1990, modified as described in Karlin and Altschul, Proc, Natl. Acad. Sci. USA 5873-5877, 1993. Such an algorism is incorporated into the NBLAST and XBLAST programs of Altschul et al., J. Mol. Biol. 215:403-410, 1990. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al., Nucleic Acids Res. 25:3389-3402, 1997. When utilizing the BLAST and Gapped BLAST programs, the default parameters of the respective programs (e.g., XBLAST and NBLAST) are used. See www.ncbi.nlm.nih.gov.

A functional variant of mAb7E (e.g., a humanized antibody) can be generated by introducing mutations in a frame region (FR) of either the V_{H} or V_{L} of mAb7E and keep their CDRs intact. It is well known that CDRs of an antibody determine its specificity. Accordingly, mutations in FRs normally would not affect antibody specificity. The CDRs and FRs of an antibody can be determined based on the amino acid sequences of its V_{H} and V_{L}. See www.bioinforg.uk/abs. The binding-specificity of the functional equivalents described herein can be examined using methods known in the art, e.g., ELISA or western-blot analysis.

Alternatively, a functional variant of mAb7E is a genetically engineered antibody containing the same V_{H} and V_{L} as mAb7E. Such a variant (e.g., a chimeric antibody or a single-chain antibody) can be prepared following methods described above.

When used for treating oral cancer, any of the anti-IL-20 antibodies described herein can be mixed with a pharmaceutically acceptable carrier to form a pharmaceutical composition. “Acceptable” means that the carrier must be compatible with the active ingredient of the composition (and preferably, capable of stabilizing the active ingredient) and not deleterious to the subject to be treated. Suitable carriers include microcrystalline cellulose, mannitol, glucose, defatted milk powder, polyvinylpyrrolidone, and starch, or a combination thereof.

To practice the cancer treatments provided in this application, the above-described pharmaceutical composition can be administered via a conventional route, e.g., orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation.

In addition, the pharmaceutical composition described above can be administered to the subject via injectable depot routes of administration such as using 1-, 3-, or 6-month depot injectable or biodegradable materials and methods.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### Example 1

### Inhibiting Oral Cancer Cell Proliferation and Migration by mAb7E

OC-3 cells, an immortalized cell line derived from a human patient with oral cancer, were seeded in wells of a 96-well culture plate (2,000 cells/well) and incubated in 200 μl DMEM medium supplemented with 10% FBS at 37° C. in a humidified atmosphere of 5% CO₂ for 24 hours. The culture medium was then replaced with 200 μl DMEM (1% FBS) supplemented with IL-20 and ³H-thymidine (Perkin-Elmer Life and Analytical Science, Knoxfield, Victoria, Australia) to a final IL-20 concentration of 25, 50 or 100 ng/ml and a final ³H-thymidine concentration of 5 μCi/ml. Quadruplicate cultures at each IL-20 concentration were conducted.

The IL-20 treated cells were incubated at 37° C. in humidified 5% CO₂ for 72 hours. The culture medium was then removed from each well and all of the wells were rinsed twice with 200 μl PBS. The cells were incubated with 50 μl PBS containing 0.25% trypsin (Sigma-Aldrich) at 37° C. for 10 minutes and the trypsinized cells were lysed by incubation in 200 μl dH₂O for 20 minutes at room temperature. DNAs were isolated from the cell lysates using glass fibre filters and a cell harvester (Skatron Instruments AS, Lier, Norway). The filters were then air-dried and punched into scintillation vials. A scintillation cocktail solution (3 ml/vial) was added into each vial, which was then vortexed for 30 to 40 seconds. The radioactivity of ³H incorporated into the DNA of OC-3 cells was determined by scintillation counting (Tricarb 2900 TR; Perkin-Elmer Life and Analytical Science). The results obtained from this study indicate that IL-20 stimulated OC-3 cell proliferation in a dose-dependent manner.

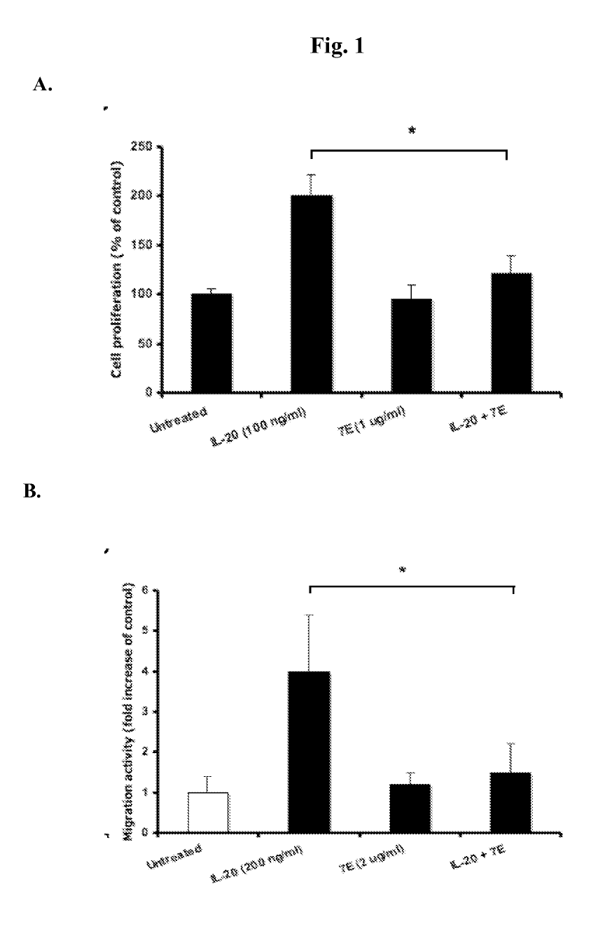

OC-3 cells were treated with IL-20 (200 ng/ml), mAb7E (2 μg/ml), or a combination of IL-20 (200 ng/ml) and mAb7E (2 μg/ml). Proliferation of the treated cells was analyzed following the method described above. As shown in FIG. 1, panel A, IL-20 stimulated OC-3 cell growth and this stimulatory effect was reversed by mAb7E. In addition, mAb7E alone suppressed OC-3 cell growth.

The effect of IL-20 in stimulating OC-3 cell migration was investigated as follows, using a Boyden camber, which contains an upper well and a lower chamber separated by a polycarbonate filter with 8 μm pores. 5000 OC-3 cells were placed in the upper well and the lower chamber was filled with DMEM medium supplemented with human IL-20 (100 or 200 ng/ml) and 0.1% FBS. The Boyden camber was incubated for 4.5 hours at 37° C. Cells attached to the bottom side of the filter were fixed with methanol and stained using a Giemsa solution (Diff-Quick; Baxter Healthcare, Deerfield, Ill.). The number of the attached OC-3 cells was determined microscopically by counting 12 randomly selected fields (at 100× magnification). The results obtained from this study indicate that IL-20 activated OC-cell migration.

To test the effect of mAb7E in suppressing OC-3 cell migration, the same Boyden chamber assay as described above was performed except that the lower chamber was filled with DMEM medium supplemented 0.1% FBS and human IL-20 (200 ng/ml), mAb7E (2 μg/ml), or a combination of IL-20 (200 ng/ml), mAb7E (2 μg/ml). The results are shown in FIG. 1, panel B. IL-20 was found to stimulate OC-3 migration, while this stimulatory effect was reversed by mAb7E. This antibody, alone, suppressed OC-3 cell migration.

### Example 2

### Treating Oral Cancer with mAb7E

OEC-M1 cancer cells, an immortalized and well-differentiated human cell line derived from an oral cancer patient, were cultured under standard conditions. The cells, suspended in PBS at a concentration of 1×10⁷/ml, were inoculated into mammary fat pads in eight—week male SCID mice, which were anesthetized with pentobarbital at 50 mg/kg body weight. The mice were then randomly assigned into 3 groups (n=6/group), each injected subcutaneously (three times in one week) with the following agents:

Group 1: PBS (vehicle control)

Group 2: a mouse control IgG (mIgG) at 4 mg/kg)

Group 3: mAb7E at 4 mg/kg

Mice free of OEC-M1 cancer cells were used as healthy controls. The size of the tumor developed on the mammary fat pad of each mouse was measured every week until the end of the experiment. An average tumor size for each group was calculated.

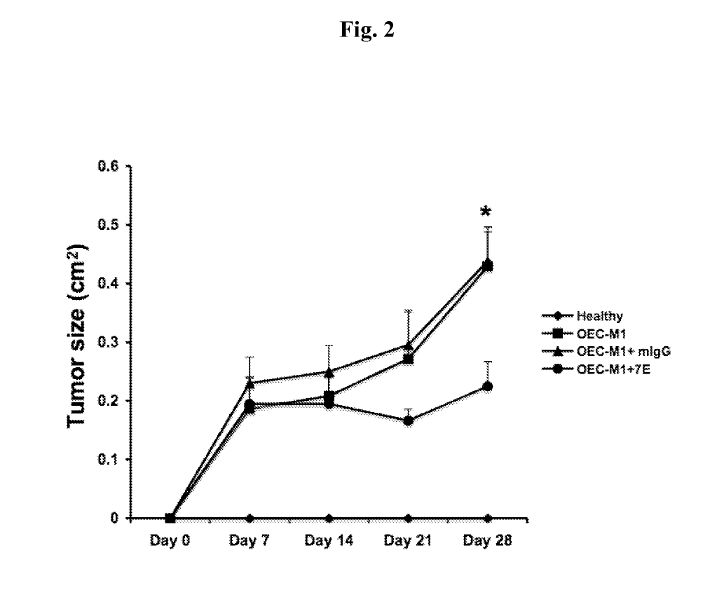

As shown in FIG. 2, the tumor size of Group 3 (means±standard deviation), treated with mAb7E, was much smaller than those of Groups 1 and 2, treated with the vehicle control and the control IgG.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for treating oral cancer, comprising administering to a subject in need thereof an effective amount of an anti-IL-20 antibody.

2. The method of claim 1, wherein the anti-IL-20 antibody is a humanized antibody, a chimeric antibody, a single-chain antibody, a naturally-occurring antibody or an antigen-binding fragment thereof.

3. The method of claim 2, wherein the anti-IL-20 antibody contains a heavy chain variable region including all of the complementarity-determining regions in SEQ ID NO:4 and a light chain variable region including all of the complementarity-determining regions in SEQ ID NO:8.

4. The method of claim 3, wherein the anti-IL-20 antibody contains a heavy chain variable region including SEQ ID NO:4 and a light chain variable region including SEQ ID NO:8.

5. The method of claim 4, wherein the anti-IL-20 antibody is a chimeric antibody or a single-chain antibody.

6. The method of claim 4, wherein the anti-IL-20 antibody is monoclonal antibody mAb7E or an antigen-binding fragment thereof.

7. The method of claim 1, wherein the subject is an oral cancer patient who has or is at risk for cancer metastasis.

8. The method of claim 7, wherein the anti-IL-20 antibody is a humanized antibody, a chimeric antibody, a single-chain antibody, a naturally-occurring antibody or an antigen-binding fragment thereof.

9. The method of claim 8, wherein the anti-IL-20 antibody contains a heavy chain variable region including all of the complementarity-determining regions in SEQ ID NO:4 and a light chain variable region including all of the complementarity-determining regions in SEQ ID NO:8.

10. The method of claim 9, wherein the anti-IL-20 antibody contains a heavy chain variable region including SEQ ID NO:4 and a light chain variable region including SEQ ID NO:8.

11. The method of claim 10, wherein the anti-IL-20 antibody is a chimeric antibody or a single-chain antibody.

12. The method of claim 11, wherein the anti-IL-20 antibody is monoclonal antibody mAb7E or an antigen-binding fragment thereof.


SEQUENCE LISTING (8 sequences)

SEQ ID NO: 1 - DNA, 1395 bp, Mus musculus
atgtacttgggactgaactatgtattcatagtttttctcttaaatggtgtccagagtgaattgaagcttgaggagtctggaggaggcttggtgcagcctggaggatccatgaaactctcttgtgctgcctctggattcacttttagtgacgcctggatggactgggtccgccagtctccagagaaggggcttgagtggattgctgaaattagaagcaaagctaataattatgcaacatactttgctgagtctgtgaaagggaggttcaccatctcaagagatgattccaaaagtggtgtctacctgcaaatgaacaacttaagagctgaggacactggcatttatttctgtaccaagttatcactacgttactggttcttcgatgtctggggcgcagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 2 - protein, 464 aa, Mus musculus
MYLGLNYVFIVFLLNGVQSELKLEESGGGLVQPGGSMKLSCAASGFTFSDAWMDWVRQSPEKGLEWIAEIRSKANNYATYFAESVKGRFTISRDDSKSGVYLQMNNLRAEDTGIYFCTKLSLRYWFFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 3 - DNA, 363 bp, Artificial sequence
  nucleotide sequence for the VH of mAb 7E heavy chain
gaattgaagcttgaggagtctggaggaggcttggtgcagcctggaggatccatgaaactctcttgtgctgcctctggattcacttttagtgacgcctggatggactgggtccgccagtctccagagaaggggcttgagtggattgctgaaattagaagcaaagctaataattatgcaacatactttgctgagtctgtgaaagggaggttcaccatctcaagagatgattccaaaagtggtgtctacctgcaaatgaacaacttaagagctgaggacactggcatttatttctgtaccaagttatcactacgttactggttcttcgatgtctggggcgcagggaccacggtcaccgtctcctca

SEQ ID NO: 4 - protein, 122 aa, Artificial sequence
  Amino acid sequence for the VH of mAb 7E heavy chain
SELKLEESGGGLVQPGGSMKLSCAASGFTFSDAWMDWVRQSPEKGLEWIAEIRSKANNYATYFAESVKGRFTISRDDSKSGVYLQMNNLRAEDTGIYFCTKLSLRYWFFDVWGAGTTVTVSS

SEQ ID NO: 5 - DNA, 720 bp, Mus musculus
atgatgagtcctgcccagttcctgtttctgttagtgctctggattcgggaaaccaacggtgattttgtgatgacccagactccactcactttgtcggttaccattggacaaccagcctccatctcttgcaagtcaagtcagagcctcttggatagtgatggaaagacatatttgaattggttgttacagaggccaggccagtctccaaagcacctcatctatctggtgtctaaactggactctggagtccctgacaggttcactggcagtggatcagggaccgatttcacactgagaatcagcagagtggaggctgaggatttgggagtttattattgctggcaaagtacacattttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctacaagtggaagattgatggcagtgaacgacaaaatggcgtcctgaacagttggactgatcagcccaaagacatcaatgtcgacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 6 - protein, 239 aa, Mus musculus
MMSPAQFLFLLVLWIRETNGDFVMTQTPLTLSVTIGQPASISCKSSQSLLDSDGKTYLNWLLQRPGQSPKHLIYLVSKLDSGVPDRFTGSGSGTDFTLRISRVEAEDLGVYYCWQSTHFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - DNA, 339 bp, Artificial sequence
  Nucleotide sequence of the VL of mAb 7E light chain
gattttgtgatgacccagactccactcactttgtcggttaccattggacaaccagcctccatctcttgcaagtcaagtcagagcctcttggatagtgatggaaagacatatttgaattggttgttacagaggccaggccagtctccaaagcacctcatctatctggtgtctaaactggactctggagtccctgacaggttcactggcagtggatcagggaccgatttcacactgagaatcagcagagtggaggctgaggatttgggagtttattattgctggcaaagtacacattttccgtggacgttcggtggaggcaccaagctggaaatcaaacgg

SEQ ID NO: 8 - protein, 114 aa, Artificial sequence
  Amino acid sequence of the VL of mAb 7E light chain
GDFVMTQTPLTLSVTIGQPASISCKSSQSLLDSDGKTYLNWLLQRPGQSPKHLIYLVSKLDSGVPDRFTGSGSGTDFTLRISRVEAEDLGVYYCWQSTHFPWTFGGGTKLEIKR